(19) United States
(12) Patent Application Publication
(10) Pub. No.: US 2014/0004170 A1
(43) Pub. Date: Jan. 2, 2014

(54) COATING OF A DRUG-ELUTING MEDICAL DEVICE

(71) Applicants: Lutz Kröhen, Berlin (DE); Andreas Voigt, Berlin (DE); Christoph Dunmann, Berlin (DE)

(72) Inventors: Lutz Kröhen, Berlin (DE); Andreas Voigt, Berlin (DE); Christoph Dunmann, Berlin (DE)

(73) Assignee: CAPSULUTION PHARMA AG, Berlin (DE)

(21) Appl. No.: 13/978,142

(22) PCT Filed: Jun. 1, 2011
(86) PCT No.: PCT/EP11/59147

§ 371 Date: Sep. 23, 2013

(30) Foreign Application Priority Data
Jun. 3, 2010 (EP) 10164854.1

(51) Int. Cl. A61L 29/16; A61L 29/08

(52) U.S. Cl. CPC A61L 29/16; A61L 29/085
USPC 424/423; 427/2.24

(57) ABSTRACT

The present invention relates to a method for preparing a drug-eluting implantable or insertable medical device comprising the steps of (i) providing a implantable or insertable medical device having a surface, (ii) depositing at least two oppositely charged polyelectrolyte layers on at least a portion of said surface to form a polyelectrolyte multilayer on the surface, and (iii) depositing one or more layers of particulate pharmaceutically active ingredient within said polyelectrolyte multilayer or on top of said polyelectrolyte multilayer. The invention also pertains to a drug-eluting implantable or insertable medical device obtained by such a method and the medical uses of such devices.

### TECHNICAL FIELD OF THE INVENTION

The present invention is in the field of medical implants and devices. Particularly, the present invention relates to implantable or insertable drug-eluting medical devices.

### BACKGROUND OF THE INVENTION

Numerous diseases do not affect the entire organism but are restricted to specific tissues, often even to very limited individual tissue areas or organ parts. Examples can be found among tumor, joint and vascular diseases.

Medical devices are frequently used for delivery of therapeutic agents. For example, an implantable or insertable medical device, such as a stent or a balloon catheter, may be provided with a polymer matrix coating layer that contains a therapeutic agent. Upon placement of the medical device at a desired location within a patient, the therapeutic agent is released from the polymer matrix and into specific tissue areas or organ parts, thereby achieving a desired therapeutic outcome.

WO 2004/028582 A1 relates to a coated balloon that releases drugs for the selective therapy of specific tissues or organ parts and to a method of manufacturing such drug-coated balloons wherein a lipophilic drug and adjuvants are applied in a solution, suspension or emulsion medium by immersion, brushing or spraying or by means of a volume measuring device on to the surface of a folded balloon, and wherein excess media and substances adhering loosely to the surface are removed. EP 2 002 847 A1 discloses an implantable device comprising a drug-releasing coating comprising at least two oppositely charged polyelectrolyte layers and at least one pharmaceutical active drug which is covalently coupled or bound to polyelectrolytes of at least one of the polyelectrolyte layers. EP 2 016 957 A1 relates to a method for coating a catheter with a multilayer of alternating polyelectrolyte layers and non-polymeric drug layers. WO 2005/089825 A2 provides a medical article comprising a ceramic or metallic region whose surface comprises a plurality of depressions, a multilayer coating region comprising multiple polyelectrolyte layers deposited over said surface and a therapeutic agent disposed beneath or within said multilayer coating region.

On example for the medical use of a drug-eluting device is the treatment of coronary in-stent restenosis with a Paclitaxel-coated balloon catheter as described by Scheller et al. in N Engl J Med (2006) 355:2113-24.

Thierry and coworkers (Biomacromolecules (2003), 4:1564-1571) describe an endovascular stent coated with a layer-by-layer technique and speculate that it might be used for drug delivery.

However, the drug-eluting medical devices of the prior art have a relatively low transfer rate of pharmaceutically active ingredient to the target tissue. This requires a relatively high loading of the surface of such devices with the pharmaceutically active ingredients.

### SUMMARY OF THE INVENTION

The present invention provides drug-eluting implantable or insertable medical devices with improved transfer properties of pharmaceutically active ingredients to the target tissue and improved release kinetics.

The present invention is in part based on the inventors' finding that layer-by-layer coatings of drug-eluting medical devices have particularly advantageous properties when the pharmaceutically active ingredient (the “drug”) is present in particulate form in or on the coating. Furthermore, the inventors found in a particular aspect that it is advantageous for the release of the coating from the medical device if the coating comprises consecutive layers of polyelectrolytes that are oppositely charged at the time and under the conditions of their deposition but which have the same net charge under physiological conditions, i.e. in situ. This may be achieved by the use of a combination of a polyelectrolyte and an amphoteric substance in the coating wherein the amphoteric substance has a different net charge at the pH conditions during coating than at the pH in the target tissue.

The invention relates in particular to a drug-eluting implantable or insertable medical device comprising
a surface, and
a multilayer coating at least on a portion of said surface, wherein the multilayer coating comprises at least two alternating layers of oppositely charged polyelectrolytes and further comprises a particulate pharmaceutically active ingredient in and/or on the multilayer coating.

In a particularly preferred embodiment of the drug-eluting implantable or insertable medical device of the invention, the coating additionally comprises a rapidly disintegrating bilayer comprising at least two layers of oppositely charged polyelectrolytes on the surface of the device or, as the case may be, on the basic layer, and below the polyelectrolyte multilayer, wherein one of the polyelectrolytes of the basic coating is an amphoteric substance that has a different net charge at the pH of deposition than at physiological pH such that the two polyelectrolytes of the basic coating have the same net charge at physiological pH and separation of the multilayer from the surface of the device is facilitated at physiological pH.

In another particularly preferred embodiment of the drug-eluting implantable or insertable medical device of the invention, the multilayer coating comprises layers of polyelectrolytes that are oppositely charged under deposition and storage conditions, but one of the polyelectrolytes changes its net charge when subjected to physiological conditions.

The present invention also relates to a method for preparing a drug-eluting implantable or insertable medical device comprising the steps of:
(i) providing a implantable or insertable medical device having a surface,
(ii) depositing at least two oppositely charged polyelectrolyte layers on at least a portion of said surface to form a polyelectrolyte multilayer on the surface, and
(iii) depositing one or more layers of particulate pharmaceutically active ingredient within said polyelectrolyte multilayer or on top of said polyelectrolyte multilayer.

Preferably, the deposition of the polyelectrolyte layers is performed at a non-physiological pH at which the two polyelectrolytes are oppositely charged and wherein one of the polyelectrolytes is an amphoteric substance that has a different net charge at a physiological pH such that the two polyelectrolytes have the same net charge at a physiological pH.

In particularly preferred embodiments of the inventive method at least one of the polyelectrolytes is an amphoteric substance.

In one preferred embodiment of the method of the invention, additionally a basic coating comprising at least two layers of oppositely charged polyelectrolytes is deposited on the surface of the device and below the polyelectrolyte multilayer, wherein one of the polyelectrolytes of the basic coating is amphoteric and has a different net charge at the pH of deposition than at physiological pH such that the two polyelectrolytes of the basic coating have the same net charge at physiological pH and separation of the multilayer from the surface of the device is facilitated at physiological pH.

In another preferred embodiment of the invention the deposition of the polyelectrolyte layers is performed at a non-physiological pH at which the two polyelectrolytes are oppositely charged and wherein one of the polyelectrolytes is an amphoteric substance that has a different net charge at a physiological pH such that the two polyelectrolytes have the same net charge at a physiological pH.

The invention also pertains to a drug-eluting implantable or insertable medical device obtained or obtainable by the method according to the invention.

Furthermore, the drug-eluting implantable or insertable medical device according to the invention may be used in the treatment of tumours, for creating open passages in the body, for the treatment of vascular diseases or circulatory disturbances, for the treatment of gynecological diseases and conditions, for the treatment of stenosis or in the prophylaxis of restenosis.

The invention, thus, relates to the use of a drug-eluting implantable or insertable medical device as described herein for providing a means for the treatment of tumours, for creating open passages in the body, for the treatment of vascular diseases or circulatory disturbances, for the treatment of gynecological diseases and conditions, for the treatment of stenosis or for the prophylaxis of restenosis.

### DESCRIPTION OF DRAWINGS

FIGS. 1 to 6 herein only provide schematic illustrations of the fundamental design of exemplary embodiments of the coatings. The proportions of the individual layers with respect to each other may vary largely. The proportions in these drawings do not necessarily reflect the proportions of the layers of coatings produced with the method of the invention. For example, the layers comprising particles of pharmaceutically active ingredient typically have a much larger thickness than the polyelectrolyte layers because of the particle size of the particles of pharmaceutically active ingredient.

FIG. 1 shows a schematic representation of the coating of a drug-eluting implantable or insertable medical device according to a particular embodiment of the invention. 11: surface of implantable or insertable medical device; 21 basic layer (optional); 31: polyelectrolyte multilayer comprising polyelectrolyte bilayers; 33: polyelectrolyte bilayer comprising two oppositely charged layers wherein one of the layers comprises a pharmaceutically active ingredient; 35: layer of first polyelectrolyte; 51: layer of second polyelectrolyte comprising the pharmaceutically active ingredient; 41: top layer (optional).

FIG. 2 shows a schematic representation of the coating of a drug-eluting implantable or insertable medical device according to another particular embodiment of the invention. 11: surface of implantable or insertable medical device; 21 basic layer (optional); 31: polyelectrolyte multilayer comprising polyelectrolyte bilayers; 33: polyelectrolyte bilayer comprising two oppositely charged layers; 35: layer of first polyelectrolyte; 36: layer of second polyelectrolyte; 51: layer comprising a pharmaceutically active ingredient and optionally a polyelectrolyte; 41: top layer (optional).

FIG. 3 shows a schematic representation of the coating of a drug-eluting implantable or insertable medical device according to another particular embodiment of the invention. 11: surface of implantable or insertable medical device; 21 basic layer (optional); 31: polyelectrolyte multilayer comprising polyelectrolyte bilayers and layers comprising a pharmaceutically active ingredient; 33: polyelectrolyte bilayer comprising two oppositely charged layers; 35: layer of first polyelectrolyte; 36: layer of second polyelectrolyte; 51: layer comprising a pharmaceutically active ingredient and optionally a polyelectrolyte. The layer (51) comprising the pharmaceutically active ingredient and the neighbouring polyelectrolyte layers (35) have opposing net charges at least during coating; 41: top layer (optional).

FIG. 4 shows a schematic representation of the coating of a drug-eluting implantable or insertable medical device according to a particular embodiment of the invention with a rapidly disintegrating polyelectrolyte bilayer (61) beneath the multilayer. 11: surface of implantable or insertable medical device; 21 basic layer (optional); 31: polyelectrolyte multilayer comprising polyelectrolyte bilayers and layers comprising a pharmaceutically active ingredient; 33: polyelectrolyte bilayer comprising two oppositely charged layers; 35: layer of first polyelectrolyte; 36: layer of second polyelectrolyte; 51: layer comprising a pharmaceutically is active ingredient and optionally a polyelectrolyte. The layer (51) comprising the pharmaceutically active ingredient and the neighbouring polyelectrolyte layers (35) have opposing net charges at least during coating. 61: rapidly disintegrating polyelectrolyte bilayer. 65: layer of first polyelectrolyte of the rapidly disintegrating polyelectrolyte bilayer; 66: layer of second polyelectrolyte of the rapidly disintegrating polyelectrolyte bilayer. The first and second polyelectrolytes of the rapidly disintegrating polyelectrolyte bilayer have different net charge at the pH of coating but the same net charge under physiological pH, i.e. in situ. Either the first (65) or the second (66) polyelectrolyte is amphoteric, i.e. has a different net charge at the pH of coating than at physiological pH; 41: top layer (optional).

FIG. 5 shows a schematic representation of the coating of a drug-eluting implantable or insertable medical device according to a particular embodiment of the invention with a multilayer of rapidly disintegrating polyelectrolyte bilayers (61). 11: surface of implantable or insertable medical device; 21 basic layer (optional); 31: polyelectrolyte multilayer comprising rapidly disintegrating polyelectrolyte bilayers and layers comprising a pharmaceutically active ingredient; 61: rapidly disintegrating polyelectrolyte bilayer. 65: layer of first polyelectrolyte of the rapidly disintegrating polyelectrolyte bilayer; 66: layer of second polyelectrolyte of the rapidly disintegrating polyelectrolyte bilayer. The first and second polyelectrolytes of the rapidly disintegrating polyelectrolyte bilayer have different net charge at the pH of coating but the same net charge under physiological pH, i.e. in situ. Either the first (65) or the second (66) polyelectrolyte is amphoteric, i.e. has a different net charge at the pH of coating than at physiological pH. 51: layer comprising a pharmaceutically active ingredient and optionally a polyelectrolyte. The layer (51) comprising the pharmaceutically active ingredient and the neighbouring polyelectrolyte layers (65) have opposing net charges at least during coating; 41: top layer (optional).

FIG. 6 shows a schematic representation of the coating of a drug-eluting implantable or insertable medical device according to a particular embodiment of the invention with a multilayer of rapidly disintegrating polyelectrolyte bilayers (61). 11: surface of implantable or insertable medical device; 21 basic layer (optional); 31: polyelectrolyte multilayer comprising rapidly disintegrating polyelectrolyte bilayers wherein one of the layers of the bilayer comprises a pharmaceutically active ingredient; 61: rapidly disintegrating polyelectrolyte bilayer. 65: layer of first polyelectrolyte of the rapidly disintegrating polyelectrolyte bilayer; 51: layer of second polyelectrolyte of the rapidly disintegrating polyelectrolyte bilayer comprising pharmaceutically is active ingredient. The pharmaceutically active ingredient either is itself a polyelectrolyte or the layer (51) comprises a polyelectrolyte in addition to the pharmaceutically active ingredient. The first and second polyelectrolytes of the rapidly disintegrating polyelectrolyte bilayer have different net charge at the pH of coating but the same net charge under physiological pH, i.e. in situ. Either the first (65) or the second (66) polyelectrolyte is amphoteric, i.e. has a different net charge at the pH of coating than at physiological pH. 41: top layer (optional).

FIG. 7 illustrates layer build-up and shows the decrease in frequency (left axis) and increase in adsorbed mass (right axis), respectively, per layer for a crystals coated with PEI-[ChonS/GelB]₁₂ at pH 2.5 (Example 3).

FIG. 8 illustrates pH-dependent desorption of polyelectrolyte layers in mass vs. time at pH 7.4 for crystals coated with PEI-[ChonS/GelB]₁₂ at pH 2.5 (Example 3).

FIG. 9 illustrates layer build-up and shows the decrease in frequency (left axis) and increase in adsorbed mass (right axis), respectively, per layer for a crystals coated with PEI-[ChonS/HSA]₁₂ at pH 2.5 (Example 4).

FIG. 10 illustrates pH-dependent desorption of polyelectrolyte layers in mass vs. time at pH 7.4 for crystals coated with PEI-[ChonS/HSA]₁₂ at pH 2.5 (Example 4).

FIG. 11 illustrates the net charge of the coated particles in terms of zeta potential during coating. Shown is the zeta potential of PEI-(ChonS/MP)₅ LBL-coating onto CaCO₃ particles (Precarb 720) at pH 3.5 to control layer build-up (Example 5).

FIG. 12 illustrates the increase of the overall concentration of MP in the multilayer coating after 2, 4, 6, 8 and 10 layers of coating. Shown is the increase in MP drug concentration during LBL-coating (PEI-(ChonS/MP)₅) onto CaCO₃ particles (Precarb 720) at pH 3.5; measurement of supernatants after coating with MP layer by UV-Vis spectroscopy; 2-fold experiment (Example 5)

FIG. 13 illustrates the release of the MP at physiological pH. Shown is the release of MP at pH 7.4 (PBS) out of CaCO₃ particles (Precarb 720) coated with LBL-coating PEI-(ChonS/MP)₅ at pH 3.5; measured by UV-Vis spectroscopy; 2-fold experiment (Example 5)

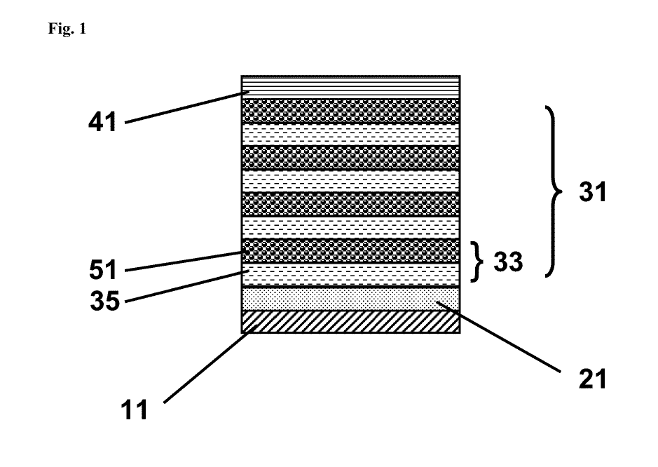

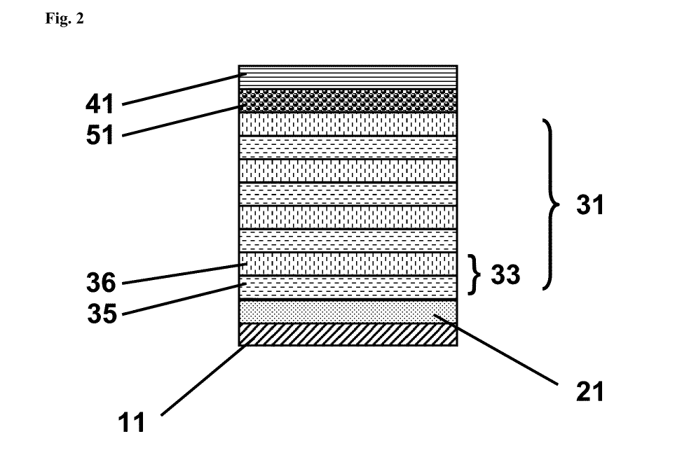

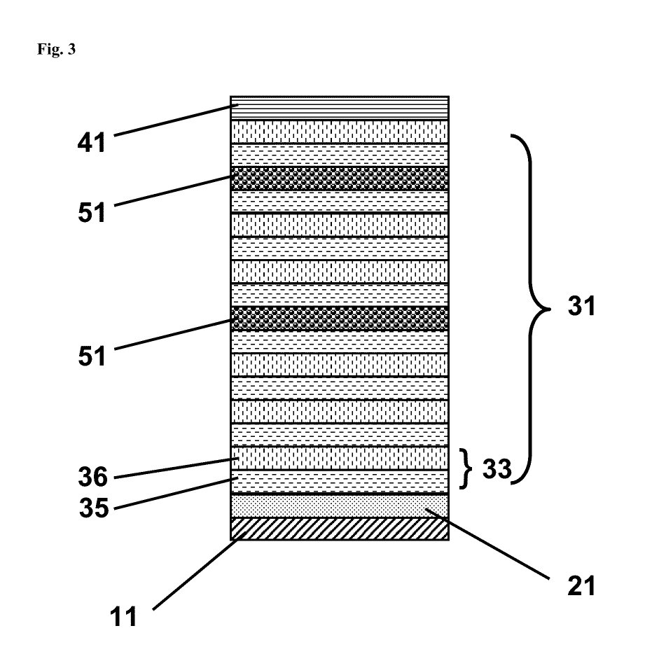

### DETAILED DESCRIPTION OF THE INVENTION

Implantable or insertable medical devices benefiting from the present invention include any medical device for which controlled release of a therapeutic agent is desired. The terms “therapeutic agent”, “drug”, “pharmaceutically active agent” and “pharmaceutically active ingredient” and other related terms may be used interchangeably herein.

Examples of such medical devices include for instance, catheters (e.g., renal or vascular catheters such as balloon catheters), guide wires, balloons, filters (e.g., vena cava filters), stents (including coronary vascular stents, cerebral, urethral, ureteral, biliary, tracheal, gastrointestinal and esophageal stents), stent grafts, cerebral aneurysm filler coils (including Guglilmi detachable coils and metal coils), vascular grafts, myocardial plugs, patches, pacemakers and pacemaker leads, heart valves, orthopedic implants, temporary implants in the mouth (e.g., temporarily crown jackets which release a pain killer), artificial implanted lenses and biopsy devices. Hence, the drug-eluting implantable or insertable medical device of the invention may for example be an expandable device and/or comprises an inflatable portion. The device may also a be removable device. Preferred examples of implantable or insertable medical device according to the invention include stents, catheters, particularly balloon catheters, pacemakers, or artificial vessels (permanent or transient) or parts of such devices such as balloons in the case of balloon catheters.

The medical devices of the present invention include medical devices that are used for either systemic treatment or for the localized treatment of any mammalian tissue or organ. Examples include tumors; organs including the heart, coronary and peripheral vascular system (referred to overall as “the vasculature”), lungs, trachea, esophagus, brain, liver, kidney, bladder, urethra and ureters, eye, intestines, stomach, pancreas, ovary, and prostate; skeletal muscle; smooth muscle; breast; dermal tissue; cartilage; and bone.

As used herein, “treatment” refers to the prevention of a disease or condition, the reduction or elimination of symptoms associated with a disease or condition, or the substantial or complete elimination a disease or condition. Preferred subjects are mammalian subjects and more preferably human subjects.

A “particulate pharmaceutically active ingredient” in the context of the present invention is a pharmaceutically active ingredient in particulate form, i.e. the pharmaceutically active ingredient is not present as single molecules in solution but aggregated as particles. Preferably the particles of pharmaceutically active ingredient herein have particle sizes of from 10 nm to 100 μm, more preferably from 100 nm to 10 μm, most preferably from 0.5 μm to 3 μm. It is also preferred that the particle size is smaller than 3 μm. It is furthermore preferred that the particle size is larger than 100 nm, more preferably larger than 500 nm. The particles of pharmaceutically active ingredient may for example be prepared by dry milling, wet milling, jet milling, spray drying, solvent evaporation methods and the like. This allows for an exact adjustment of the particle size or selection of defined particle size fraction by sieving or other appropriate classification methods.

In one aspect the present invention relates to a method for preparing a drug-eluting implantable or insertable medical device comprising the steps of:
(i) providing a implantable or insertable medical device having a surface,
(ii) depositing at least two oppositely charged polyelectrolyte layers on at least a portion of said surface to form a polyelectrolyte multilayer on the surface, and
(iii) depositing one or more layers of particulate pharmaceutically active ingredient within said polyelectrolyte multilayer or on top of said polyelectrolyte multilayer.

With the methods according to the invention, a drug-eluting implantable or insertable medical device is coated with a polyelectrolyte multilayer (31) comprising a pharmaceutically active ingredient. The polyelectrolyte multilayer of the coating typically consists of one or more polyelectrolyte bilayers (33). A polyelectrolyte bilayer in this context is the combination of a layer of a first polyelectrolyte (35) with a layer of a second polyelectrolyte (36), wherein the first and the second polyelectrolyte have opposite net charges under the conditions (particularly the pH) of the formation of the multilayer, i.e. one polyelectrolyte is an anion and the other is a cation at the conditions of deposition. The coating of the medical device may for example comprise a basic layer (21) directly on the surface to be coated (11), the polyelectrolyte multilayer (31) on the basic layer (21) and a top layer (41) on the polyelectrolyte multilayer (31). However, basic layer (21) and top layer (41) are both entirely optional. The basic layer (21) may be applied in order to mediate the adhesion of the multilayer to the surface of the medical device. The top layer (41) may serve as a protection of the multilayer (31) from external influences such as pressure or chemicals.

Polyelectrolyte multilayers can be assembled using various known layer-by-layer techniques. Layer-by-layer techniques involve coating various substrates using charged polymeric (polyelectrolyte) materials via electrostatic, self-assembly. In the layer-by-layer technique, a first polyelectrolyte layer having a first net charge is typically deposited on an underlying substrate, followed by a second polyelectrolyte layer having a second net charge that is opposite in sign to the net charge of the first polyelectrolyte layer, and so forth. The charge on the outer layer is reversed upon deposition of each sequential polyelectrolyte layer or at least the net charge is substantially reduced. To the extent that the surface of the medical device does not have an inherent net surface charge, a surface charge may be provided. For example, where the surface to be coated is conductive, the surface charge can be provided by applying an electrical potential to the same. Once a first polyelectrolyte layer is established in this fashion, a second polyelectrolyte layer having a second net charge that is opposite in sign to the net charge of the first polyelectrolyte layer can readily be applied, and so forth. As another example, a surface charge can be provided by exposing the surface to be coated to a charged amphiphilic substance. Amphiphilic substances include any substance having hydrophilic and hydrophobic groups. Where used, the amphiphilic substance should have at least one electrically charged group to provide the substrate surface with a net electrical charge. Therefore, the amphiphilic substance that is used herein can also be referred to as an ionic amphiphilic substance. Amphiphilic polyelectrolytes can be used as ionic amphiphilic substances. For example, a polyelectrolyte comprising charged groups (which are hydrophilic) as well as hydrophobic groups, such as polyethylenimine (PEI) or poly(styrene sulfonate) (PSS), can be employed. Cationic and anionic surfactants can also be used as amphiphilic substances. Cationic surfactants include quaternary ammonium salts (R4N+X″), for example, didodecyldimethylammonium bromide (DDDAB), alkyltrimethylammonium bromides such as hexadecyltrimethylammonium bromide (HDTAB), dodecyltrimethylammonium bromide (DTMAB), myristyltrimethylammonium bromide (MTMAB), or palmityl trimethylammonium bromide, or N-alkylpyridinium salts, or tertiary amines (R3NH+X″), for example, cholesterol-3β-N-(dimethyl-aminoethyl)-carbamate or mixtures thereof, wherein X″ is a counter-anion, e.g. a halogenide. Anionic surfactants include alkyl or olefin sulfate (R—OSO3M), for example, a dodecyl sulfate such as sodium dodecyl sulfate (SDS), a lauryl sulfate such as sodium lauryl sulfate (SLS), or an alkyl or olefin sulfonate (R—SO3M), for example, sodium-n-dodecyl-benzene sulfonate, or fatty acids (R—COOM), for example, dodecanoic acid sodium salt, or phosphoric acids or cholic acids or fluoro-organics, for example, lithium-3-[2-(perfluoroalkyl)ethylthiojpropionate or mixtures thereof, where R is an organic radical and M is a counter-cation. Hence, the method may in particular embodiments comprise the step of depositing a layer of an amphiphilic substance to said portion of the surface before depositing the polyelectrolyte multilayer. Such a layer is herein designated “basic layer” (21).

In other embodiments, a surface charge is provided by adsorbing cations (e.g., protamine sulfate, polyallylamine, polydiallyldimethylammonium species, polyethyleneimine, chitosan, gelatin, spermidine, albumin, among others) or anions (e.g., polyacrylic acid, sodium alginate, polystyrene sulfonate, eudragit, gelatin (gelatin is an amphoteric polymer, hence it fits in both categories depending how it is being prepared), hyaluronic acid, carrageenan, chondroitin sulfate, carboxymethylcellulose, among others) to the surface to be coated as a first charged layer. Although full coverage may not be obtained for the first layer, once several layers have been deposited, a full coverage should ultimately be obtained, and the influence of the substrate is expected to be negligible. The species for establishing surface charge can be applied to the ceramic or metallic region by a variety of techniques. These techniques include, for example, spraying techniques, dipping techniques, roll and brush coating techniques, techniques involving coating via mechanical suspension such as air suspension, ink jet techniques, spin coating techniques, web coating techniques and combinations of these processes. Alternatively or additionally, an activation of the surface can be performed, for instance by chemical etching with e.g. a H₂O/HCl/H₂O₂ mixture and/or a H₂O/NH₃/H₂O₂ mixture or plasma etching. This results in temporary charges on the surface which in turn promote the adsorption of polyelectrolytes to the surface. An exemplary protocol for chemical etching is provided in the examples.

Once a sufficiently charged substrate is obtained, it can be coated with a layer of an oppositely charged polyelectrolyte. Multilayers are formed by repeated treatment with alternating oppositely charged polyelectrolytes, i.e., by alternating treatment with cationic and anionic polyelectrolytes. The polymer layers self-assemble by means of electrostatic layer-by-layer deposition, thus forming a multilayered polyelectrolyte coating over the surface to be coated.

Polyelectrolytes are polymers having charged (e.g., ionically dissociable) groups. Usually, the number of these groups in the polyelectrolytes is so large that the polymers in dissociated form (also called polyions) are water-soluble. Depending on the type of dissociable groups, polyelectrolytes are typically classified as polyacids and polybases. When dissociated, polyacids form polyanions, with protons being split off. Polyacids include inorganic, organic and bio-polymers. Examples of polyacids are polyvinylphosphoric acids, polyvinylsulfonic acids, polyvinylsulfonic acids, polyvinylphosphonic acids and polyacrylic acids. Examples of the corresponding salts, which are also called polysalts, are polyvinylphosphates, polyvinylsulfates, polyvinylsulfonates, polyvinylphosphonates and polyacrylates. Polybases contain groups which are capable of accepting protons, e.g., by reaction with acids, with a salt being formed. Examples of polybases having dissociable groups within their backbone and/or side groups are polyallylamine, polyethylimine, polyvinylamine and polyvinylpyridine. By accepting protons, polybases form polycations. Quaternary ammonium groups are also preferred cationic groups in the context of the present invention. For example polydiallyl dimethyl ammonium chloride (PDADMAC) is a very strong cationic charged polyelectrolyte.

Suitable polyelectrolytes according to the invention include those based on biopolymers, for example, alginic acid, gummi arabicum, nucleic acids, pectins and proteins, chemically modified biopolymers such as carboxymethyl cellulose and lignin sulfonates, and synthetic polymers such as polymethacrylic acid, polyvinylsulfonic acid, polyvinylphosphonic acid and polyethylenimine. Linear or branched polyelectrolytes can be used. Using branched polyelectrolytes can lead to less compact polyelectrolyte multilayers having a higher degree of wall porosity. Polyelectrolyte molecules can be crosslinked within or/and between the individual layers, e.g. by crosslinking amino groups with aldehydes, for example, to increase stability. However, it is preferred in the context of the present invention that the polyelectrolytes are not cross-linked. Furthermore, amphophilic polyelectrolytes, e.g. amphiphilic block or random copolymers having partial polyelectrolyte character, can be used to affect permeability towards polar small molecules. Such amphiphilic copolymers consist of units having different functionality, e.g. acidic or basic units, on the one hand, and hydrophobic units, on the other hand (e.g., polystyrenes, polydienes or polysiloxanes), which can be present in the polymer as blocks or distributed statistically. Suitable polyelectrolytes include low-molecular weight polyelectrolytes (e.g., polyelectrolytes having molecular weights of a few hundred Daltons) up to macromolecular polyelectrolytes (e.g., polyelectrolytes of biological origin, which commonly have molecular weights of several million Daltons). Preferably herein, at least one of the used polyelectrolytes has a molecular weight of below 100 kDa, preferably below 10 kDa. Specific examples of polycations include protamine sulfate polycations, poly(allylamine) polycations (e.g., poly(allylamine hydrochloride) (PAH)), polydiallyldimethylammonium (PDADMAC) polycations, polyethyleneimine (PEI) polycations, chitosan polycations, spermidine polycations and albumin polycations. Specific examples of polyanions include poly(styrenesulfonate) polyanions (e.g., poly(sodium styrene sulfonate) (PSS)), polyacrylic acid polyanions, sodium alginate polyanions, hyaluronic acid polyanions, carrageenan polyanions, chondroitin sulfate polyanions, carboxymethylcellulose polyanions and albumin polyanions.

By using polyelectrolytes that are biodisintegrable, the release of the therapeutic agent can be further controlled based on the rate of disintegration of the polyelectrolyte layers. Moreover, as indicated above, implantable or insertable medical articles containing a biodisintegrable multilayer polyelectrolyte coating leave behind only the underlying ceramic or metallic structure once the therapeutic agent is released from the medical article. As used herein, a “biodisintegrable” material is a material which undergoes dissolution, degradation, resorption and/or other disintegration processes upon administration to a patient. Preferred examples of biodisintegrable polyelectrolytes include protamine sulfate, gelatin, spermidine, albumin, carrageenan, chondroitin sulfate, heparin, other polypeptides and proteins, and DNA, among others. As with species for establishing surface charge (described above), the polyelectrolyte layers can be applied to the surface to be coated by a variety of techniques including, for example, spraying techniques, dipping techniques, roll and brush coating techniques, techniques involving coating via mechanical suspension such as air suspension, inkjet techniques, spin coating techniques, web coating techniques and combinations of these processes. Preferably, herein, the polyelectrolyte layers are applied by spraying, brushing or by immersion of the surface to be coated into a solution comprising the respective polyelectrolyte. The layers comprising the pharmaceutically active ingredient can equally be applied by the same techniques, i.e. preferably spraying techniques, dipping techniques, roll and brush coating techniques, techniques involving coating via mechanical suspension such as air suspension, inkjet techniques, spin coating techniques, web coating techniques and combinations of these processes, most preferably by spraying, brushing or by immersion. In a preferred embodiment a layer comprising the pharmaceutically active ingredient is applied from a suspension. Such a suspension may not only comprise the pharmaceutically active ingredient but may also comprise one or more polymers, preferably one or more polyelectrolytes.

Tables 1 to 3 list preferred cationic, anionic and amphoteric polyelectrolytes, respectively.

TABLE 1
Examples of cationic polyelectrolytes

| | molecular weight |
|---|---|
| Polymers | (MW) |
| Protamine (Prot) | ca. 4 800 Da |
| Chitosan (Chi) | wide range |
| Polyethylenimine (PEI) | 10->300 kDa |
| Poly-L-arginine (PLArg) | 10->300 kDa |
| Poly-L-lysine (PLL) | 10->300 kDa |
| Spermine | 238-348 Da |
| Spermidine | 145-255 Da |

TABLE 2
Examples of anionic polyelectrolytes

| Polymers | MW |
|---|---|
| Carboxymethylcellulose | wide range |
| Hyaluronic acid | 1.6-3.3 MDa |
| Chondroitin sulfate | 15-50 kDa |
| Heparin | 3-30 kDa |
| Alginate acid | wide range |
| Carrageenan | ι, κ, λ, |
| Gums (Xynthan, Acacia . . . ) | wide range |

TABLE 3
Examples of amphoteric polyelectrolytes

| Substance | MW | Behavior |
|---|---|---|
| Serum Albumin | 66 kDa | positive < IEP 4.7 > negative |
| Gelatin A | wide range | positive < IEP 8-9 > negative |
| Gelatin B | wide range | positive < IEP 4.8-5.4 > negative |
| Collagen | 130 kDa | positive in neutral/acid solution |
| | | positive and negative matrices |

According to the method of the invention, preferably between 2 and 1000, more preferably between 2 and 250 layers of polyelectrolytes are applied and a polyelectrolyte multilayer of alternating charge is formed.

One or more layers of the polyelectrolyte multilayer may comprise the pharmaceutically active ingredient. For example, every second, third, fourth, fifth, sixth, seventh, eighth, ninth or tenth polyelectrolyte layer of one particular charge may comprise particulate pharmaceutically active ingredient. Alternatively, in cases were the pharmaceutically active ingredient is itself a polyelectrolyte, one or more or even all of the layers of polyelectrolyte of one particular charge may be a layer of the pharmaceutically active ingredient.

The pharmaceutically active ingredient may itself be a charged polymer, e.g. a polyelectrolyte under the conditions of coating.

The polyelectrolyte multilayer may have different regions. It might, for example, be that a region is present which does not comprise layers of pharmaceutically active ingredient but only layers of alternating polyelectrolytes, while in another region of the multilayer layers of pharmaceutically active ingredient and/or layers comprising the pharmaceutically active ingredient are present. The number of layers of pharmaceutically active ingredient or comprising the pharmaceutically active ingredient for example depends on the desired loading of the coating and the desired release profile. In general, any sequence of layers may be applied in the multilayer as long as layers of different net charge alternate at the conditions of coating. Various different exemplary embodiments are shown in the appended figures.

As described herein above, the polyelectrolytes can, inter alia, be synthetic polymers, biopolymers such as polypeptides, proteins, polysaccharides, oligosaccharides, nucleic acids and derivates of biopolymers such as chemically modified biological polymers. The oppositely charged polyelectrolytes may be polyanions and polycations. Preferably, the polyanion is selected from the group consisting of chondroitin sulphate (ChonS), heparin (Hep), poly-L-glutamic acid (PLG) Carboxymethylcellulose (CMC), Hyaluronic acid (Hya), albumin such as human serum albumin (HSA), gelatine type B (GelB) or a mixture of any of these. The polycation is preferably selected from the group consisting of protamine sulphate (PS), chitosan (Chit), polyethyleneimine (PEI), Spermin, Poly-L-lysine (PLL), Poly-L-arginine, gelatine type A (GelA) or a mixture of any of these.

Table 4 lists preferred combinations of polyelectrolytes for stable polyelectrolyte multilayers (PEM).

TABLE 4
Examples of stable polyelectrolyte combinations, stable PEM

| Cationic Layers | Anionic Layers | Behavior |
|---|---|---|
| Chitosan | Chondroitin sulfate, | in vivo stable >60 min |
| | Heparin, or | |
| | Carrageenan | |
| Protamine, PLL, PArg, | Chondroitin sulfate, | in vivo stable >60 min |
| PEI, PAH, or | Heparin, | |
| PDADMAC | Carrageenan, | |
| | Carboxymethylcellulose, | |
| | Alginate acid, | |
| | Hyaluronic acid, PSS, or | |
| | PAA | |

The net charge of a polyelectrolyte may depend on the pH of the surrounding solution. For example some polyelectrolytes may be amphoteric. An amphoteric substance is a substance that can react as either an acid or base. Amphoteric substances have an isoelectric point (pI or IEP), i.e. a pH at which they have no net charge and are thus neutral. Above the pI the amphoteric substance is deprotonated and thus has a negative net charge. Below the pI the amphoteric substance is protonated and thus has a positive net charge. Hence, whether a given amphoteric polyelectrolyte is a polyanion or a polycation depends on the surrounding pH. The present inventors have in a specific embodiment exploited this fact by using an amphoteric polyelectrolyte layer that has a different net charge during coating, i.e. oppositely charged than the neighbouring layers, than under physiological conditions in situ. When a coating comprising such an amphoteric polyelectrolyte layer is brought to physiological pH, i.e. by implanting or inserting the coated medical device into a subject, the net charge of the amphoteric polyelectrolyte changes, resulting in rapid disintegration of the polyelectrolyte layer. This, in turn, leads to an improved release of the pharmaceutically active ingredient to the surrounding (target) tissue. In the context of drug-eluting medical devices, a rapid and/or efficient release of the pharmaceutically active ingredient to the surrounding (target) tissue is sometimes preferred, particularly in the case of insertable devices such as catheters, particularly balloon catheters.

Hence, in particularly preferred embodiments of the invention at least one of the polyelectrolytes is an amphoteric substance.

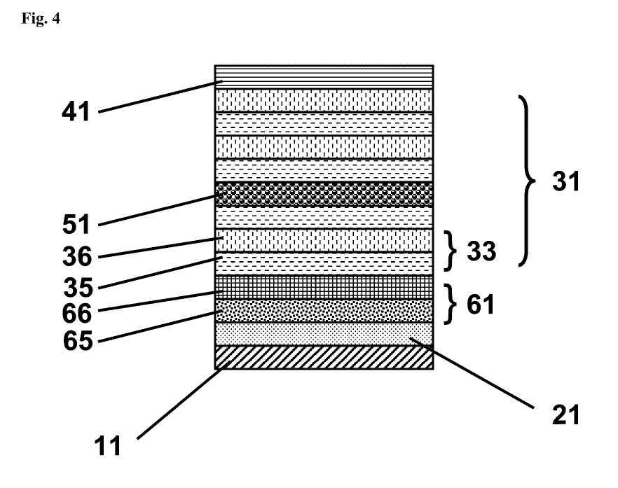

In some preferred embodiments, additionally a coating (61) comprising at least two layers of oppositely charged polyelectrolytes (65, 66) is deposited on the surface of the device (11) and below the polyelectrolyte multilayer (31), wherein one of the polyelectrolytes of the basic coating is an amphoteric substance that has a different net charge at the pH of deposition than at physiological pH such that the two polyelectrolytes of the basic coating have the same net charge at physiological pH and separation of the multilayer from the surface of the device is facilitated at physiological pH. Such an embodiment is illustrated in appended FIG. 4.

“Physiological conditions”, particularly “physiological pH”, herein refers to the conditions, particularly the pH, at the place to which the implantable or insertable medical device is implanted or inserted, i.e. the conditions, particularly the pH, in situ. In the case of blood vessels, the pH is typically between 7.35 and 7.45, preferably around 7.4. The osmolarity in whole blood plasma is typically between 250 and 330 mosmol/kg, mostly between 275 and 299 mosmol/kg.

In other preferred embodiments, the deposition of the polyelectrolyte layers is performed at a non-physiological pH at which the two polyelectrolytes are oppositely charged and wherein one of the polyelectrolytes is amphoteric and has a different net charge at a physiological pH such that the two polyelectrolytes have the same net charge at a physiological pH. Preferably, the application of the pharmaceutically active ingredient is then performed at a pH at which the two polyelectrolytes do not have the same net charge.

Table 5 lists preferred combinations of a polyelectrolyte with an amphoteric substance for the formation of rapidly disintegrating polyelectrolyte bilayers or polyelectrolyte multilayers.

TABLE 5
Examples of rapidly disintegrating polyelectrolyte combinations,
rapidly disintegrating PEM

| Cationic Layers | Anionic Layers | Behavior |
|---|---|---|
| Albumin (IEP ca. 4.6-4.7) | Chondroitin sulfate | Instable in vivo, |
| | Heparin | disintegrate <60 min |
| | Carrageenan | @pH 7.4 |
| Gelatin B (IEP ca. 4.8-5.4) | Chondroitin sulfate | Instable in vivo, |
| | Heparin | disintegrate <60 min |
| | Carrageenan | @pH 7.4 |
| Amphoteric polyelectrolyte | Anionic material | Instable in vivo, |
| (IEP <6) | (charged below IEP | disintegrate <60 min |
| | of used amphoteric | @pH 7.4 |
| | polyelectrolyte) | |
| Cationic material (charged | Amphoteric | Instable in vivo, |
| above IEP of used | polyelectrolyte | disintegrate <60 min |
| amphoteric polyelectrolyte) | (IEP >8) | @pH 7.4 |

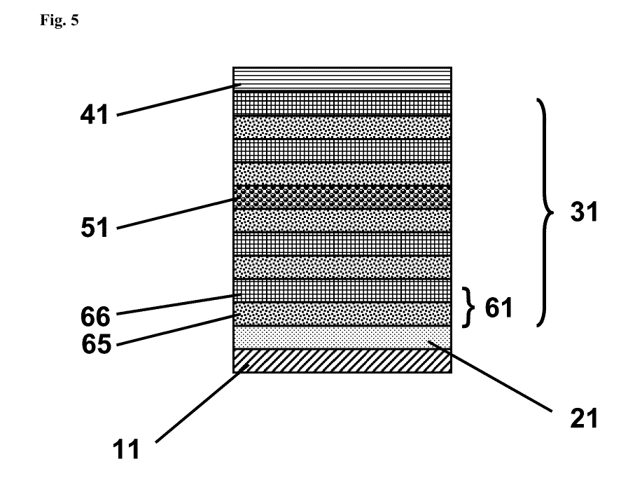

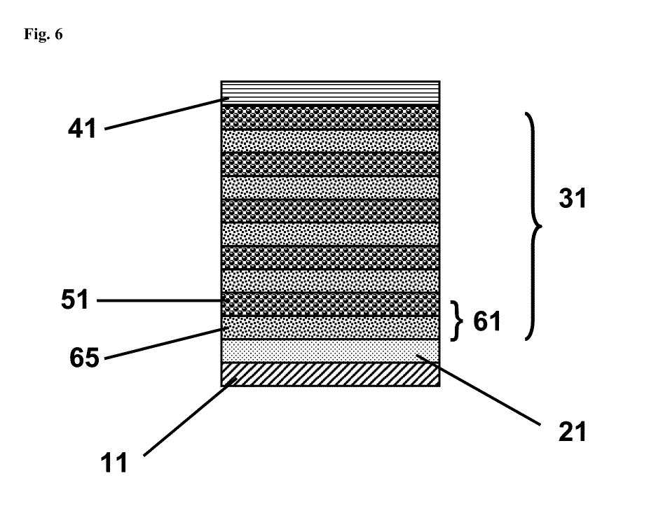

Hence, in one preferred embodiment, the rapidly disintegrating bilayer (e.g. as in FIG. 4) or the rapidly disintegrating polyelectrolyte multilayer (e.g. see FIGS. 5 and 6) is formed of layers of an amphoteric polyelectrolyte with an IEP below 6 and an anionic material which is charged below the IEP of the amphoteric polyelectrolyte used. In an alternative preferred embodiment the rapidly disintegrating bilayer (e.g. as in FIG. 4) or the rapidly disintegrating polyelectrolyte multilayer (e.g. see FIGS. 5 and 6) is formed of layers of an amphoteric polyelectrolyte with an IEP above 8 and an cationic material which is charged above the IEP of the amphoteric polyelectrolyte used.

The pharmaceutically active ingredient in the context of the present invention is for instance a substance for inhibiting cell proliferation or inflammatory processes, an anti-cancer drug, an antibiotic, a growth factor, a hormone, a cytostatic, an immunosuppressant or an antioxidant.

The pharmaceutically active ingredient may e.g. be selected from the group consisting of Paclitaxel and other taxanes, Sirolimus (Rapamycin) and related substances such as Zotarolimus, Everolimus and Biolimus A9, Tacrolimus and related substances such as Docetaxel, corticoids, sexual hormones and related substances, statins, epothilones, Probucol, prostacyclins or angiogenesis inducers. In some particular embodiments the pharmaceutically active agent is a substance for enhancing tissue growth, e.g. endothelial or endometrical tissues such as growth factors or hormones.

In a particular embodiment of the method according to the invention, additionally a layer comprising one or more substances that influence the sliding quality of the device or reduce blood coagulation is deposited on top of the multilayer. The additional layer may for example comprise an anticoagulant, e.g. heparin. Such a layer herein is also designated “top layer” (41).

According to the invention additionally also a layer comprising one or more substances that facilitate dissolution of the multilayer upon insertion or implantation of the device may be deposited on top of the multilayer. Such substances include for example enzymes that cleave polyelectrolytes, e.g. polysaccharides. Such enzymes are for example chitinase, esterase, peptidase, and lysozyme. Such a layer herein is also designated “top layer” (41).

The invention further relates to a drug-eluting implantable or insertable medical device obtained or obtainable by a method as described above.

In yet another aspect, the present invention relates to a drug-eluting implantable or insertable medical device comprising
a surface, and
a multilayer coating at least on a portion of said surface, wherein the multilayer coating comprises at least two alternating layers of oppositely charged polyelectrolytes and further comprises a particulate pharmaceutically active ingredient in and/or on the multilayer coating.

Preferably, the coating of the drug-eluting implantable or insertable medical device according to the invention comprises a multilayer of between 2 and 1000, preferably between 2 and 250 layers of polyelectrolytes of alternating charge.

The polyelectrolytes in the multilayer coating of the drug-eluting implantable or insertable medical device may for example be selected from the group consisting of synthetic polymers, biopolymers such as polypeptides, proteins, polysaccharides, oligosaccharides, nucleic acids and derivates of biopolymers such as chemically modified biological polymers.

The oppositely charged polyelectrolytes may be polyanions and polycations. The polyanion may for example be selected from the group consisting of chondroitin sulphate (ChonS), heparin (Hep), poyl-L-glutamic acid (PLG) Carboxymethylcellulose (CMC), Hyaluronic acid (Hya), albumin such as human serum albumin (HSA), gelatine type B (GelB) or a mixture of any of these. The polycation may for example be selected from the group consisting of protamine sulphate (PS), chitosan (Chit), polyethyleneimine (PEI), Spermin, Poly-L-lysine (PLL), Poly-L-arginine, gelatine type A (GelA) or a mixture of any of these.

Particularly preferred combinations of polycations and polyanions include (polycation/polyanion):
protamine sulphate (PS)/human serum albumin (HSA)
human serum albumin (HSA)/chondroitin sulphate (ChonS)
human serum albumin (HSA)/heparin (Hep)
chitosan (Chit)/heparin (Hep)
gelatine type A (GelA)/gelatine type B (GelB).

The coating of the medical device according to the invention may additionally comprise a layer of an amphiphilic substance directly below the multilayer, the so-called “basic layer” (21).

In a preferred embodiment of the medical device of the invention, at least one of the polyelectrolytes is amphoteric.

In a particular embodiment of the invention, the multilayer coating of the medical device additionally comprises a basic coating comprising at least two layers of oppositely charged polyelectrolytes is deposited on the surface of the device and below the polyelectrolyte multilayer, wherein one of the polyelectrolytes of the basic coating is an amphoteric substance that has a different net charge at the pH of deposition than at physiological pH such that the two polyelectrolytes of the basic coating have the same net charge at physiological pH and separation of the multilayer from the surface of the device is facilitated at physiological pH.

In another very preferred embodiment of the invention, the coating comprises layers of polyelectrolytes that are oppositely charged under deposition and storage conditions, wherein one of the polyelectrolytes changes its net charge under physiological conditions.

The surface of the implantable or insertable medical device may for example be a plastic, metal glass or ceramic surface. The surface can e.g. be polyamide-based.

The implantable or insertable medical device may for example be a stent or a catheter, preferably a balloon catheter, pacemaker, artificial vessel (permanent or transient) or a part thereof. In particularly preferred embodiments, the implantable or insertable medical device is a balloon catheter and the surface or a portion of the surface of the balloon is coated with the method of the invention.

The invention further relates to a drug-eluting implantable or insertable medical device obtained or obtainable by a method as described above.

In a particularly preferred embodiment, the implantable or insertable medical device contains a balloon. It is preferred that the implantable or insertable medical device is a balloon catheter and the surface of the balloon is coated.

The pharmaceutically active ingredient in the context of the present invention is for instance a substance for inhibiting cell proliferation or inflammatory processes, an anti-cancer drug, an antibiotic, a growth factor, a hormone, a cytostatic, an immunosuppressant or an antioxidant.

The pharmaceutically active ingredient may e.g. be selected from the group consisting of Paclitaxel and other taxanes, Sirolimus (Rapamycin) and related substances such as Zotarolimus, Everolimus and Biolimus A9, Tacrolimus and related substances such as Docetaxel, corticoids, sexual hormones and related substances, statins, epothilones, Probucol, prostacyclins or angiogenesis inducers.

The drug-eluting implantable or insertable medical device according to the invention, preferably comprises between 2 and 1000, more preferably between 2 and 250 layers of polyelectrolytes in the coating. The polyelectrolyte layers each have preferably a thickness of from 0.1 to 50 nm, more preferably from 2 to 20 nm. The coating has preferably an overall thickness of from 2 nm to 500 μm, more preferably from 100 nm to 50 μm, most preferably from 0.4 μm to 10 μm.

In one particular embodiment of the implantable or insertable medical device, the surfaces is additionally coated with one or more substances that influence the sliding quality of the device or reduce blood coagulation. The additional layer may for example comprise an anticoagulant, e.g. heparin.

According to the invention, the coating may additionally also comprise a layer comprising one or more substances that facilitate dissolution of the multilayer upon insertion or implantation of the device. Such substances include for example enzymes that cleave polyelectrolytes, e.g. polysaccharides. Such enzymes are for example chitinase, esterase, peptidase, and lysozyme.

In yet another aspect, the present invention pertains to a drug-eluting insertable medical device comprising
a surface, and
a multilayer coating at least on a portion of said surface, wherein the multilayer coating comprises at least two alternating layers of oppositely charged polyelectrolytes and further comprises a pharmaceutically active ingredient in and/or on the coating,
   wherein more than 15%, preferably more than 30%, more preferably more than 50%, most preferably more than 70% of the total amount of pharmaceutically active ingredient in and/or on the coating is transferred to the surrounding target tissue upon insertion into a patient. Preferably, between 15% and 75%, more preferably between 30 and 70% of the total amount of pharmaceutically active ingredient in and/or on the coating is transferred to the surrounding target tissue upon insertion into a patient.

Furthermore, the drug-eluting implantable or insertable medical device according to the invention may be used in the treatment of tumours, for creating open passages in the body, for the treatment of vascular diseases or circulatory disturbances, for the treatment of gynecological diseases and conditions, for the treatment of stenosis or in the prophylaxis of restenosis.

The invention, thus, relates to the use of a drug-eluting implantable or insertable medical device as described herein for providing a means for the treatment of tumours, for creating open passages in the body, for the treatment of vascular diseases or circulatory disturbances, for the treatment of gynecological diseases and conditions, for the treatment of stenosis or for the prophylaxis of restenosis.

Once the medical devices of the present invention are contacted with a subject (e.g., a human subject), the pharmaceutically active ingredient is released from the same. The release profile will depend upon a number of factors including: (a) the characteristics of the pharmaceutically active ingredient, including polarity and the molecular size, (b) the manner in which the medical device is contacted with the subject, (c) if present, the type of the rapidly disintegrating layer, and (d) the number and type of the individual polyelectrolyte layers that are selected (as noted above, biodisintegrable polyelectrolyte layers are particularly beneficial, in that one is potentially left with a bare surface subsequent to biodisintegration). Also the particle size and charge (in terms of the zeta potential) of the pharmaceutically active ingredient influences the transfer and release of the pharmaceutically active ingredient to its target tissue.

The following examples illustrate particular embodiments and aspects of the present invention. However, they are not limiting the scope of the invention.

### EXAMPLES

### Example 1

### Stable Layer-by-Layer (LbL) Coating of Balloons with Polyelectrolytes and Direct Loading of Paclitaxel (PTx) from Aqueous Dispersion into PE Multilayer

Purification and Pre-Treatment of Balloons:
40 balloons were placed in a glass staining trough containing a mixture of 143 ml demineralized water, 28.6 ml hydrogen peroxide and 28.6 ml ammonium hydroxide;
the glass staining trough was placed in a water bath and heated;
after reaching a temperature of 60° C. the trough remained in the water bath for another 30 min at this temperature;
subsequently the balloons were washed three times with pure water (3 glass beakers were filled with water, each balloon was carefully pulled through the water)

LbL Coating of Balloons:
before coating the balloons were dried and fixed on a holder
the coating was performed in glass staining troughs
the troughs were each filled with 200 ml of the respective PE solution (Polyethylenimin (PEI) 0.2g/l, 154 mM NaCl, Gelatine A (GelA) 0.2 g/l 154 mM NaCl, Gelatine B (GelB) 0.2 g/l 154 mM NaCl); subsequently the balloons were immerged for 1 min into the respective trough for the coating with the first layer;
subsequently the balloons were washed three times in pure water (3 troughs of water);
then the balloons were transferred to the next trough comprising the next PE solution (1 min coating);
the washing and coating steps were repeated as required and according to the desired sequence of PE layers; each coating step lasted 1 min;
for storage the balloons were dried.

Loading of the Coated Balloons with Paclitaxel:
Preparation of Paclitaxel solution:
   Dispersion comprising Paclitaxel (concentration of Paclitaxel (c_{PTx})=0.5 mg/ml) and albumin (concentration of albumin=0.2 g/l) (PTx-Alb)
Inclusion of Paclitaxel into the LbL-coating:
   the PTx incorporation was performed in glass staining troughs
   the troughs were each filled with the respective PTx dispersion
   balloons were immersed into the Paclitaxel solution for 5 min according to the sequence of coating (after every 5^{th} polymer layer);
   subsequently the balloons were removed from the Paclitaxel solution and transferred to a second trough containing pure water for 5 sec (washing);
   for storage the balloons were dried.
   determination of Paclitaxel amount on balloons by extraction with organic solvent and quantitative analysis using HPLC

Sequence of Coating:

PEI-(GelB/GelA)₁₀-PTx-Alb-[(GelA/GelB)_{2.5}-(PTx-Alb)]₁₉-(GelA/GelB)_{2.5}
Paclitaxel content of coated balloons:
   determined total Paclitaxel loading (HPLC, 1^{st} to 3^{rd} extraction): 78 μg

### Example 2

### Stable Layer-by-Layer (LbL) Coating of Balloons with Polyelectrolytes and Direct Loading of Paclitaxel (PTx) from Organic Dispersion into PE Multilayer

Purification and Pre-Treatment of Balloons:
40 balloons were placed in a glass staining trough containing a mixture of 143 ml demineralized water, 28.6 ml hydrogen peroxide and 28.6 ml ammonium hydroxide;
the glass staining trough was placed in a water bath and heated;
after reaching a temperature of 60° C. the trough remained in the water bath for another 30 min at this temperature;
subsequently the balloons were washed three times with pure water (3 glass beakers were filled with water, each balloon was carefully pulled through the water)

LbL Coating of Balloons:
before coating the balloons were dried and fixed on a holder;
the coating was performed in glass staining troughs
the troughs were each filled with 200 ml of the respective PE solution (Polyethylenimin (PEI) 0.2g/l, 154 mM NaCl, Gelatine A (GelA) 0.2 g/l 154 mM NaCl, Gelatine B (GelB) 0.2 g/l 154 mM NaCl); subsequently the balloons were immerged for 1 min into the respective trough for the coating with the first layer;
subsequently the balloons were washed three times in pure water (3 troughs of water);
then the balloons were transferred to the next trough comprising the next PE solution (1 min coating);
the washing and coating steps were repeated as required and according to the desired sequence of PE layers; each coating step lasted 1 min;
for storage the balloons were dried.

Loading of the Coated Balloons with Paclitaxel:
Preparation of Paclitaxel solution:
   Dispersion comprising Paclitaxel (concentration of Paclitaxel (c_{PTx})=0.5 mg/ml) in Methylcyclohexane (PTx-MCH))
Inclusion of Paclitaxel into the LbL-coating:
   the PTx incorporation was performed in glass staining troughs
   the troughs were each filled with the respective PTx dispersion
   balloons were immersed into the Paclitaxel solution for 5 min according to the sequence of coating (after every 4^{th} polymer layer);
   subsequently the balloons were removed from the Paclitaxel solution and transferred to a second trough containing pure MCH for 5 sec (washing);
   for storage the balloons were dried.
   determination of Paclitaxel amount on balloons by extraction with organic solvent and quantitative analysis using HPLC

Sequence of Coating:

PEI-(GelB/GelA)₁₀-PTx-MCH-[(GelB/GelA)₂-(PTx-MCH)]₁₉-(GelB/GelA)₂
Paclitaxel content of coated balloons:
   determined total Paclitaxel loading (HPLC, 1^{st} to 3^{rd} extraction): 50 μg

Examples 3 to 5 illustrate the pH dependent release of polyelectrolyte multilayers at physiological pH. Crystals and calcium carbonate particles are used as models for surfaces.

### Example 3

### Build-Up of Chondroitin Sulphate/Gelatine B Polyelectrolyte Layers and Desorption vs. pH

Example 3 illustrates an embodiment in which a surface (here: surface of crystals) are coated with polyelectrolyte multilayer layer (LbL: layer-by-layer). The layer is subsequently released at a physiological pH.

Materials: Layer Build-Up
PEI-[ChonS/GelB]12 at pH 2.5
PE solution 1: Polyethylenimine (PEI) (0.1 g/l) pH 2.5 containing 0.154 M NaCl
PE solution 2: Chondroitin sulphate (ChonS) (0.1 g/l) pH 2.5 containing 0.154 M NaCl
PE solution 3: Gelatine B (GelB) (0.1 g/l) pH 2.5 containing 0.154 M NaCl
Washing water: MilliQ-Water adjusted to pH 2.5

Materials: Desorption of Layers at pH 7.4
Phosphate buffer (PBS) with Tween20 at pH 7.4

Equipment:

Quartz Crystal Micro Balance (QCM-D)

Pre-Treatment of Crystals:

The crystals to be coated were treated with polymer solution 1 (basic layer)

LbL Coating:
after pre-treatment the crystals were coated with polymer solution 2 (anionic charge) at pH 2.5 for 2 min followed by a washing sequence with water at pH 2.5
subsequently the crystals were rinsed with polymer solution 3 (cationic charge) at pH 2.5 for 2 min followed by a washing sequence with water at pH 2.5
the washing and coating steps were repeated as required and according to the desired sequence of layers

Sequence of Coating:

PEI-[ChonS/GelB]₁₂

Desorption of PE Layers at pH 7.4:
rinse of crystals with PBS pH 7.4 solution for 1 min
residence time=29 min
total investigation time=16 hrs

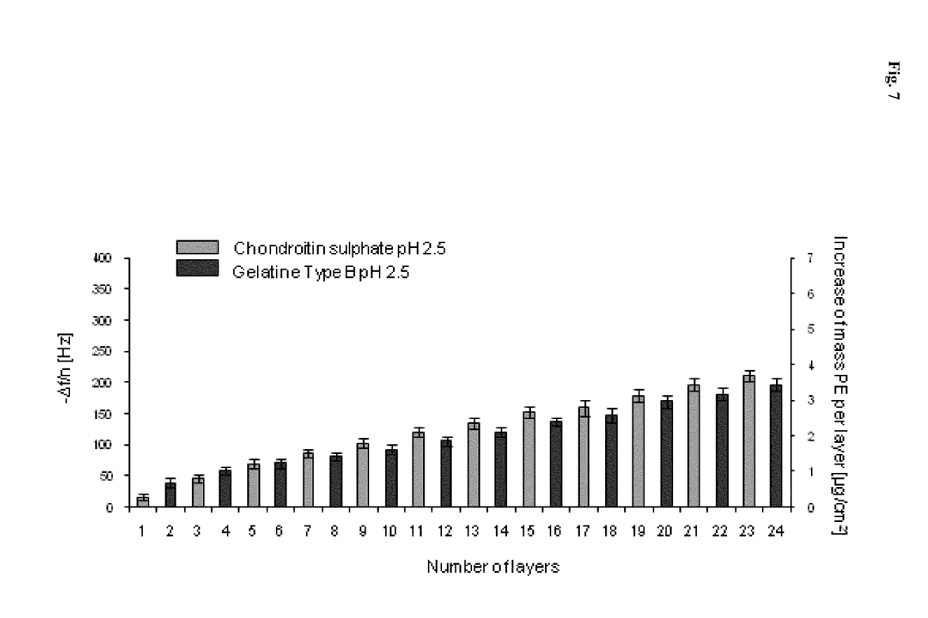

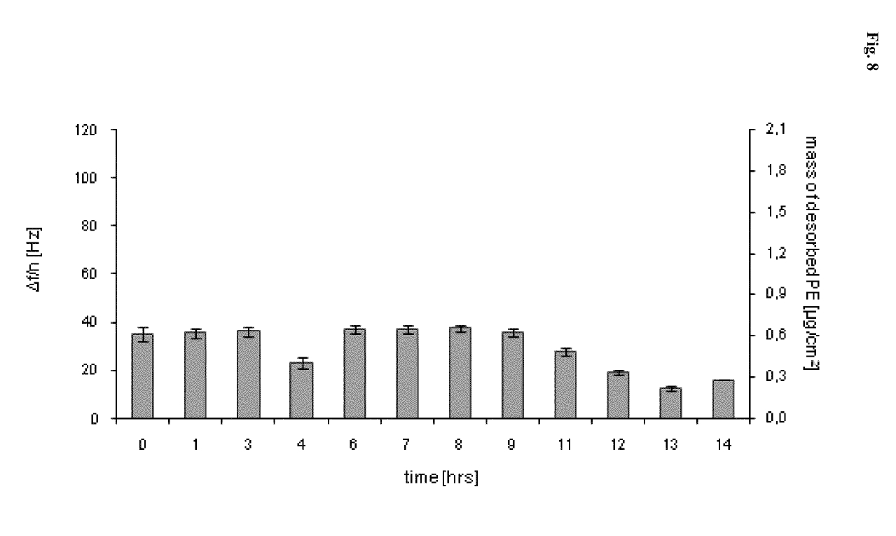

FIG. 7 and FIG. 8 show the progression of layer build-up and pH-dependent layer desorption, respectively.

### Example 4

### Build-Up of Chondroitin Sulphate/Human Serum Albumin Polyelectrolyte Layers and Desorption vs. pH

Materials: Layer Build-Up
PEI-[ChonS/HSA]12 at pH2.5
PE solution 1: Polyethylenimine (PEI) (0.1 g/l) pH 2.5 containing 0.154 M NaCl
PE solution 2: Chondroitin sulphate (ChonS) (0.1 g/l) pH 2.5 containing 0.154 M NaCl
PE solution 3: Human Serum Albumin (HSA) (0.1 g/l) pH 2.5 containing 0.154 M NaCl
Washing water: MilliQ-Water adjusted to pH 2.5

Materials: Desorption of Layers at pH 7.4
Phosphate buffer with Tween20 at pH 7.4

Equipment: Quartz Crystal Micro Balance (QCM-D)

Pre-Treatment of Crystals:
to crystals to be coated were treated with polymer solution 1 (basic layer)

LbL Coating:
after pre-treatment the crystals were coated with polymer solution 2 (anionic charge) at pH 2.5 for 2 min followed by a washing sequence with water at pH 2.5
subsequently the crystals were rinsed with polymer solution 3 (cationic charge) at pH 2.5 for 2 min followed by a washing sequence with water at pH 2.5
the washing and coating steps were repeated as required and according to the desired sequence of layers

Sequence of Coating:

PEI-[ChonS/HSA]₁₂

Desorption of PE Layers at pH 7.4:
rinse of crystals with PBS pH 7.4 solution for 1 min
residence time=29 min
total investigation time=16 hrs

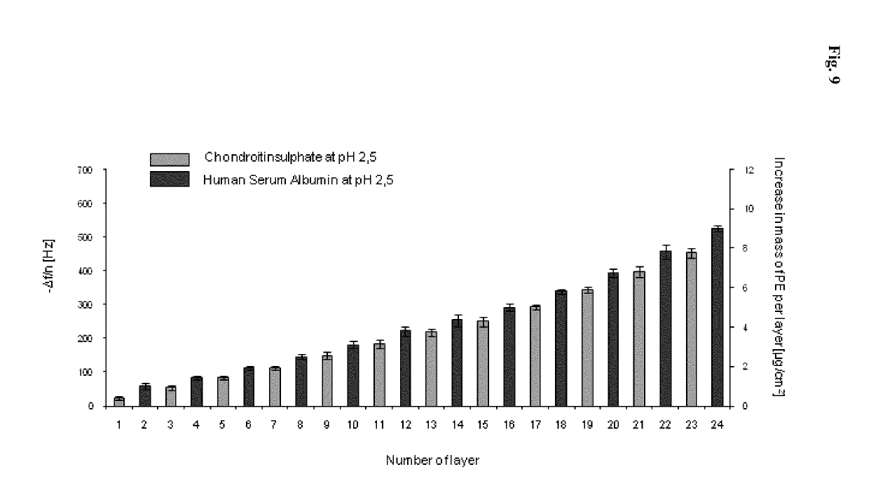

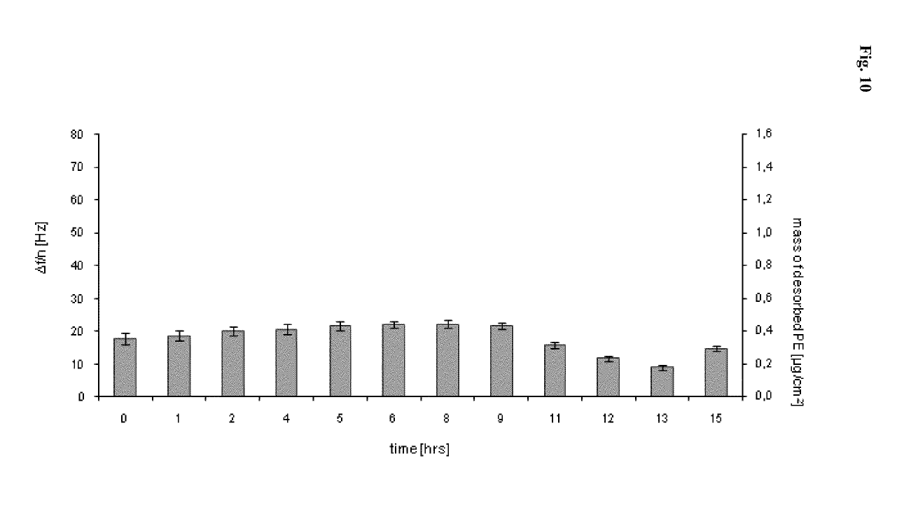

FIG. 9 and FIG. 10 show the progression of layer build-up and pH-dependent layer desorption, respectively.

### Example 5

### Coating of CaCO₃ Particles at pH 4 with Chondroitin Sulphate and a Model Protein at pH 3.5 and Release at pH 7.4

Materials
CaCO₃ particles (Precarb 720)
Polymer solution 1: containing 4 mg/ml Polyethylenimine (PEI) incl. 154 mM, pH 3.5 (cationic charged).
Polymer solution 2: containing 4 mg/ml Chondroitin sulphate (ChonS) incl. 154 mM NaCl, pH 3.5 (anionic charged).
Polymer solution 3: containing 0.8 mg/ml model protein (MP), pH 4 (cationic charged)
Washing: water pH 3.5 (adjusted with HCl/NaOH)

The model protein (MP) is a model for a therapeutic protein, i.e. a drug

Pre-Treatment of Particles:
particles to be coated (PreCarb 720) were suspended in polymer solution 1 with a final particle concentration of 25 mg/ml in suspension

LbL Coating:
the coating was performed in Eppendorf tubes of 2 ml volume
after pre-treatment the dispersion was centrifuged and the remaining particles were 3 times washed with washing water pH 3.5
subsequently polymer solution 2 was added and the particles were resuspended and coated for 10 min
subsequently the dispersion was centrifuged and the remaining particles were 3 times washed with washing water pH 3.5
subsequently polymer solution 3 was added and the particles were resuspended and coated for 10 min
the washing and coating steps were repeated as required and according to the desired sequence of layers; each coating step lasted 10 min;

Sequence of Coating:

PEI-(ChonS-MP)₅

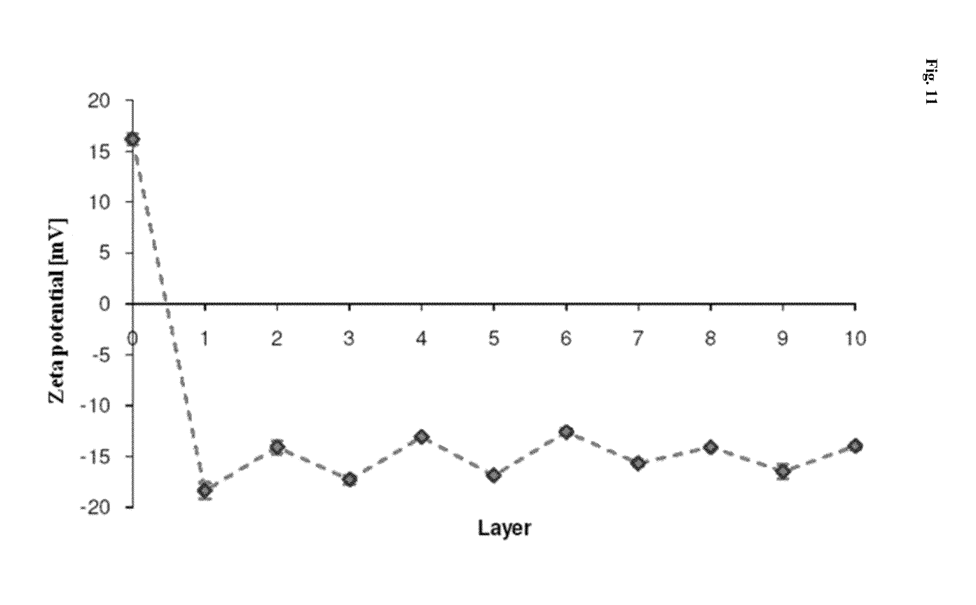

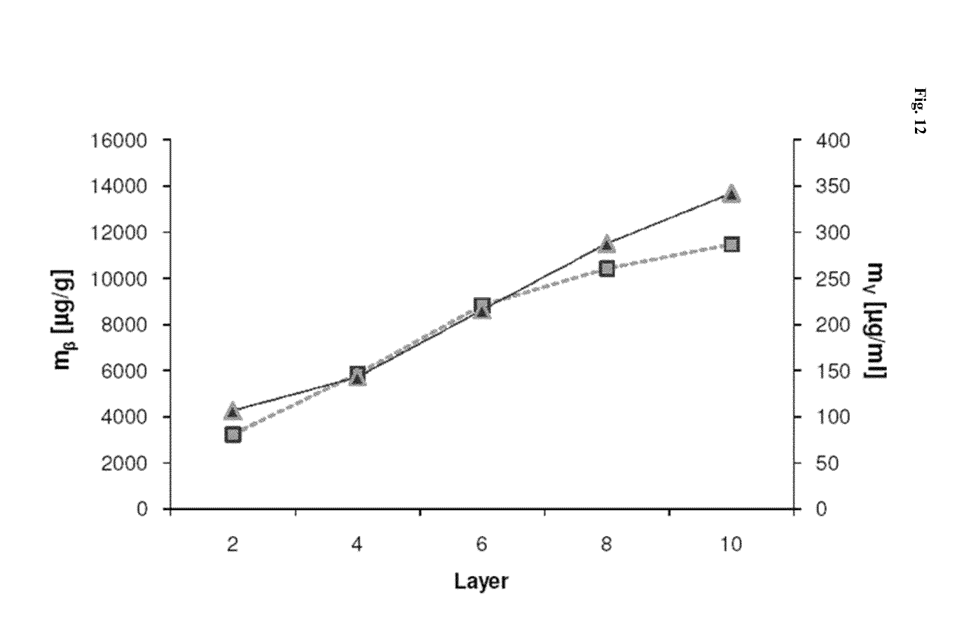

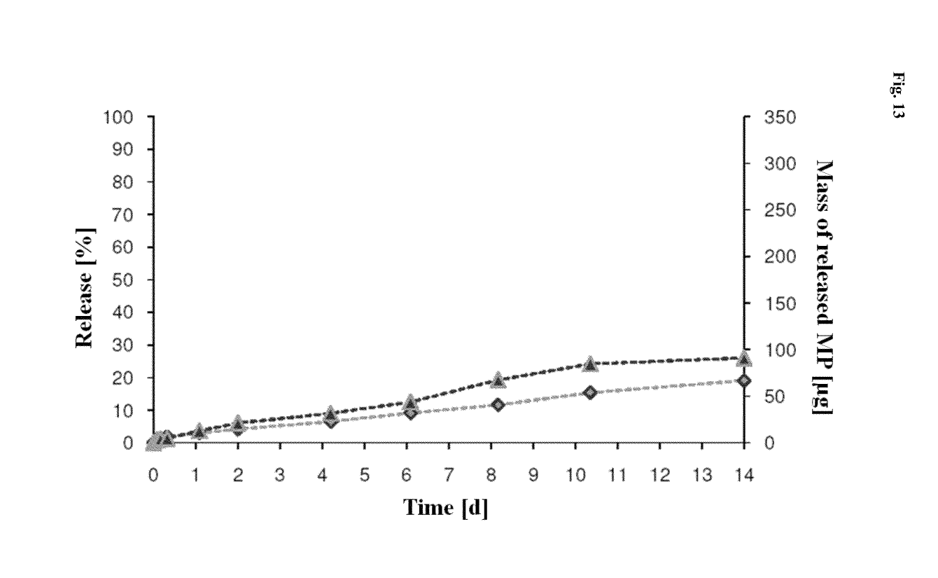

FIG. 11 illustrates the net charge of the coated particles in terms of zeta potential during coating. FIG. 12 illustrates the increase of the overall concentration of MP in the multilayer coating after 2, 4, 6, 8 and 10 layers of coating. FIG. 13 illustrates the release of the MP at physiological pH.

1. A method for preparing a drug-eluting implantable or insertable medical device comprising:
(i) providing an implantable and/or insertable medical device comprising a surface,
(ii) depositing at least two oppositely charged polyelectrolyte layers on at least a portion of said surface to form a polyelectrolyte multilayer on the surface, and
(iii) depositing at least one layer of particulate pharmaceutically active ingredient within said polyelectrolyte multilayer and/or on top of said polyelectrolyte multilayer.

2. The method according to claim 1, wherein deposition of the polyelectrolyte layers is performed at a non-physiological pH at which the two polyelectrolytes are oppositely charged and wherein at least one polyelectrolyte is an amphoteric substance which comprises a different net charge at a physiological pH such that the two polyelectrolytes comprise the same net charge at a physiological pH.

3. The method according to claim 1, wherein at least one of the two oppositely charged polyelectrolyte layers comprises the particulate pharmaceutically active ingredient.

4. The method according to claim 1, wherein the particulate pharmaceutically active ingredient comprises a particle size of from 10 nm to 100 μm, optionally from 0.5 μm to 3 μm.

5. The method according claim 1, wherein the particulate pharmaceutically active ingredient is a charged polymer under conditions of coating.

6. The method according to claim 1, wherein the oppositely charged polyelectrolytes are at least one polyanion and at least one polycation, and wherein the polyanion is selected from the group consisting of chondroitin sulphate (ChonS), heparin (Hep), poyl-L-glutamic acid (PLG) Carboxymethylcellulose (CMC), Hyaluronic acid (Hya), albumin optionally comprising human serum albumin (HSA), gelatine type B (GelB) and/or a mixture thereof.

7. The method according to claim 1, wherein the oppositely charged polyelectrolytes are at least one polyanion and at least one polycation, and wherein the polycation is selected from the group consisting of protamine sulphate (PS), chitosan (Chit), polyethyleneimine (PEI), Spermin, Poly-L-lysine (PLL), Poly-L-arginine, gelatine type A (GelA) and/or a mixture thereof.

8. The method according to claim 1, wherein the pharmaceutically active ingredient is a substance for inhibiting cell proliferation or inflammatory process, an anti-cancer drug, an antibiotic, a growth factor, a hormone, a cytostatic, an immunosuppressant and/or an antioxidant.

9. A drug-eluting implantable and/or insertable medical device comprising:
a surface, and
a multilayer coating at least on a portion of said surface, wherein the multilayer coating comprises at least two alternating layers of oppositely charged polyelectrolytes and further comprises a particulate pharmaceutically active ingredient in and/or on the multilayer coating.

10. The drug-eluting implantable and/or insertable medical device according to claim 9, wherein at least one layer of multilayer coating comprises an amphoteric substance which is oppositely charged to a polyelectrolyte in a neighbouring layer under deposition and storage conditions but comprises the same net charge as said polyelectrolyte in the neighbouring layer when subjected to physiological conditions.

11. The drug-eluting implantable and/or insertable medical device according to claim 9, wherein at least one alternating layer comprise the particulate pharmaceutically active ingredient.

12. The drug-eluting implantable and/or insertable medical device according to claim 9, wherein the pharmaceutically active ingredient is a polyelectrolyte.

13. The drug-eluting implantable and/or insertable medical device according to claim 9, wherein at least one layer of the pharmaceutically active ingredient comprise a polyelectrolyte.

14. The drug-eluting implantable and/or insertable medical device according to claim 9, wherein at least one polyelectrolyte layer of one charge of the multilayer is a layer of particulate pharmaceutically active ingredient and/or comprises the particulate pharmaceutically active ingredient.

15. The drug-eluting implantable and/or insertable medical device according to claim 9, capable of being used in treatment of tumours, for creating an open passage in a body, for treatment of vascular diseases and/or circulatory disturbances, for treatment of gynecological disease and/or conditions, for treatment of stenosis and/or in prophylaxis of restenosis.
